(19) United States
(12) Patent Application Publication
(10) Pub. No.: US 2014/0276246 A1
(43) Pub. Date: Sep. 18, 2014

(54) SYSTEM AND METHOD FOR TATTOO REMOVAL

(71) Applicant: Stephen E. Feldman, New York, NY (US)

(72) Inventor: Stephen E. Feldman, New York, NY (US)

(21) Appl. No.: 13/838,153

(22) Filed: Mar. 15, 2013

(51) Int. Cl. A61N 5/06; A61N 7/00

(52) U.S. Cl. CPC A61N 5/0616; A61N 7/00
USPC 601/2; 607/90

(57) ABSTRACT

The specification relates to a tattoo removal device that includes a light panel and a control panel for controlling the light panel. The light panel houses an ultra-bright light emitting diode (LED). The LED can have an intensity of about 5,000-20000 mcd and a viewing angle of 8-130 degrees and a primary wavelength between 400-940 nm. The intensity, the viewing angle and the primary wavelength can be dependent on a size and a color of a tattooed area. The light panel can continuously apply an energy output from the light panel directly over a tattooed area for a specified period of time resulting in degradation of the tattoo ink.

### RELATED APPLICATIONS

This application is a continuation-in-part of U.S. patent application Ser. No. 12/381,143 and claims the benefit of provisional application Ser. No. 61/068,369, filed Mar. 7, 2008, provisional application Ser. No. 61/941,173, filed Sep. 26, 2008, non-provisional application Ser. No. 13/573,624 filed Sep. 28, 2012 and non-provisional application Ser. No. 13/694,223 filed Nov. 8, 2012.

### BACKGROUND

The subject matter described herein relates to an ultra-bright-led-induced tattoo removal system and method. There are a variety of medical procedures and techniques used to remove tattoos. For example, dermabrasion is one such technique. Dermabrasion slices off or abrades the skin in which the tattoo lies. This procedure is highly invasive and often produces scars. This technique also has a tendency to leave behind pigments which lie in skin layers not removed that appear as a dark shade through the new skin.

Another technique, called a “split-skin graft,” involves the tangential excision of the tattoo area and covers the area with a skin graft. This procedure cuts out the visible tattoo area and leaves intact an underlying skin layer. The procedure is usually performed while a patient is under general anesthesia. The open area is covered with split skin and saved from unnecessary scar formation by use of compression bandages.

Another technique involves the use of lasers and pulsed radiation. These techniques also have many disadvantages. One disadvantage is that the procedure produces “speckles” on the skin due to the high power density of the light beam. The light beam can also cause significant local heating and destruction of tissues that do not contain tattoo ink. To counteract this damage heat must be removed to prevent tissue damage but this wastes a majority of the light beam's power. Another disadvantage is that these procedures involve the use of light that has a short duty cycle and specific wavelength and is thus not absorbed by some colors of tattoo ink. Another disadvantage is that the procedures cannot treat large surface areas and focuses on a very small area. In order for a tattoo to be removed, a patient must undergo many hours of sometimes painful treatment which increases with the size of the tattoo. If a large tattoo is to be removed, the tattoo treatments can be expensive. Also, these light beams can cause reactions in certain chemicals used in the inks leading to permanent darkening.

### SUMMARY

An ultra bright LED induced tattoo removal procedure can use an ultra bright LED that has no duty cycle and provides constant energy output. In use, the ultra bright LED procedure allows a cool light to penetrate through the outer skin of a subject without damaging the outer skin and effectively remove tattoo ink. Additionally, the ultra bright LED procedure can treat a large tattooed area in a single treatment.

In one implementation, an apparatus comprising: a light panel housing at least one ultra-bright light emitting diode (LED), the LED having an intensity of about 15,000-20000 mcd and a viewing angle of 8-30 degrees and a primary wavelength between 400-940 nm, the intensity, the viewing angle and the primary wavelength being dependent on a size and a color of a tattooed area, the light panel continuously applying an energy output from the light panel directly over the tattooed area for a specified period of time resulting in degradation of the tattoo ink; and a control panel for controlling the light panel, wherein the light panel is used to apply a treatment of light on a tattooed area of a subject for tattoo removal, the light panel generating a light beam that penetrates an epidermis of the subject without damaging the epidermis by overheating and enters a dermis of the subject in which tattoo ink resides and results in (a) minimal to no absorption by melanin and hemoglobin of the subject and (b) little to no heat being generated on the epidermis of the subject while generating heat on the tattoo ink thereby causing increased molecular motion and bond deformation of the tattoo ink.

Additionally, the light beam can be approximately equal to or greater than size of the tattooed area. And the control panel can include a timer for controlling the duration of a treatment. The control panel can also include a printed circuit board for controlling the timer and an intensity of the light panel. In use, L-Arginine, an immune response modifier compound or an immune response modifier compound containing L-Arginine can be applied to the tattooed area before treatment begins.

In another implementation, a method comprising the steps of: positioning a light panel a specific distance from a tattooed area, the light panel housing at least one ultra-bright light emitting diode (LED), the LED having an intensity of about 15,000-20000 mcd and a viewing angle of 8-30 degrees and a primary wavelength between 400-940 nm, the intensity, the viewing angle and the primary wavelength being dependent on a size and a color of a tattooed area; generating a light beam from the light panel, the light beam penetrating an epidermis of a subject without damaging the epidermis by overheating and enters a dermis of the subject in which tattoo ink resides and results in (a) minimal absorption by melanin and hemoglobin of the subject and (b) little to no heat being generated on the epidermis of the subject while generating heat on the tattoo ink; continuously applying the energy output from the light panel directly over the tattooed area for a specified period of time; and causing increased molecular motion and bond deformation of the tattoo ink resulting in degradation of the tattoo ink and effectively removing the tattoo.

Before positioning the light panel over the tattooed area, L-arginine or a mixture of L-arginine with an immune response modifier can be applied to the tattooed skin region. The immune response modifier can be a chemical selected from the group consisting of: imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydronaphthyridine amine, an oxazoloquinoline amine, a thiazoloquinoline amine, an oxazolopyridine amine, a thiazolopyridine amine, an oxazolonaphthyridine amine, a thiazolonaphthyridine amine, and a 1H-imidazodimer fused to a pyridine amine, a quinoline amine, a tetrahydroquinoline amine, a naphthyridine amine, and a tetrahydronaphthyridine amine.

In another implementation, an apparatus for applying a treatment of light on a tattooed area of a subject for tattoo removal, the apparatus comprising: an LED panel, the LED panel including at least one ultra bright LED, the LED having an intensity of about 5,000-20000 mcd and a viewing angle of 8-130 degrees and a primary wavelength between 400-940 nm, the intensity, the viewing angle and the primary wavelength being dependent on a size and a color of the tattooed area, the LED panel producing a continuous light beam; and a control panel for controlling the at least one ultra-bright LED, wherein the apparatus generates a light beam that penetrates an epidermis of the subject without damaging the epidermis by overheating and enters a dermis of the subject in which tattoo ink resides.

Additionally, the ultra-bright LED panel can be approximately equal to or greater than the size of the tattooed area and/or slightly concave.

### BRIEF DESCRIPTION OF THE DRAWINGS

FIG. 1 is a perspective view of a light panel constructed in accordance with the disclosed technology;

FIG. 2 is a perspective view of a light panel constructed in accordance with the disclosed technology;

FIG. 3 is a perspective view of a light panel constructed in accordance with the disclosed technology;

FIG. 4 is a block diagram showing various components which are used along with the device constructed in accordance with the disclosed technology;

FIG. 5 shows an implementation of the disclosed technology that uses a flexible neck in accordance with the disclosed technology; and

FIG. 6 is a perspective view of a light panel constructed in accordance with the disclosed technology.

### DETAILED DESCRIPTION

Although specific terms are used in the following description for sake of clarity, these terms are intended to refer only to particular structure of the invention selected for illustration in the drawings, and are not intended to define or limit the scope of the invention.

During tattoo applications, a subject's skin cells consume and store tattoo particles. More specifically, tattoo ink contains carbon particles that are suspended in water. When the tattoo ink is introduced to the skin through a needle, the water diffuses. The ink itself then spreads into the surrounding tissue cells and embeds into the skin.

The disclosed technology found using certain energies and wavelengths of light can destroy the bonds that hold tattoo ink together. In operation, a light device uses the energy contained in a light beam so that the energy is absorbed by the tattoo ink dyes. This absorbed energy results in an increased stretching, vibration and bending of the bonds which hold the dye (ink) molecules together. Ultimately, these bond stresses cause bond deformation with resulting bond failure.

For tattoo removal, an ultra bright LED with high energy output can be used. The ultra-bright LED is capable of emitting a pure color in a narrow frequency range. The color emitted from the ultra-bright LED is identified by peak wavelength (lpk) and measured in nanometers (nm). Different LED chip technologies emit light in specific regions of the visible light spectrum and produce different intensity levels. Intensity is a measure of the time-averaged energy flux or amount of light striking a given area for LEDs this is measured in terms of lumens while for a LED lighting apparatus it is measured in lux (lumens/sq. meter). Ultra bright LEDs have a brightness or luminance intensity of 5,000 to 20,000 mcd with a beam angle of 8-130 degrees which equates to a luminance flux of 0.05 to 75 lumen.

LED light output varies with the type of chip, encapsulation, efficiency of individual wafer lots and other variables. The amount of light emitted from an ultra-bright LED is quantified by a single point, on-axis luminous intensity value (lv). LED intensity is specified in terms of millicandela (mcd). MCD or Millicandela is used to denote the brightness of an LED. The higher the mcd number, the brighter the light the LED emits. Ultra bright LED's have mcd ratings that vary between 5,000 and 20,000 mcd with beam angles of 8 to 130 degrees.

LED viewing angle is a function of the LED chip type and the epoxy lens that distributes the light. View angle degree, also referred to as directivity, or the directional pattern of a LED light beam is measured in degrees. The expressed degree dictates the width of the light beam and also controls to some extent, the light intensity of a LED. View angles range from 8 to 160 degrees, and are provided through the use of optics, e.g., special lenses made to collimate light into a desired view angle. The highest luminous intensity (mcd rating) does not equate to the highest visibility. The light output from an LED chip is very directional. A higher light output is achieved by concentrating the light in a tight beam. Generally, the higher the mcd rating, the narrower the viewing angle.

Another factor is the ultra bright LED's wavelength. Nanometers or nm are used to measure the wavelengths of light. The lower the wavelength, e.g., 400 nm, the bluer and stronger the light source. Longer wavelengths above 600 nm are red. Above 680 nm, they fail into the infra Red category, which is colorless to our eyes. White LEDs have no specific wavelength. They are measured by the color of white against the chromaticity scale.

The frequency of light used to destabilize the bonds in tattoo inks depends upon the composition of the ink and its color. Additional considerations are absorption by the subject's tissue cells. For example, melanin and hemoglobin have maximum absorptions below 600 nm, i.e., maximum absorption for melanin is 335 nm and for hemoglobin is 310 nm.

In use, the primary wavelength range may be between 400 to 940 nm. The primary wavelengths are carefully chosen so that (a) there is minimal to no absorption by melanin and hemoglobin of a subject and little to no heat is generated on the epidermis of the subject and (b) enough heat is generated so that tattoo ink residing in a dermis of the subject is irradiated sufficiently to cause increased molecular motion and bond deformation of the tattoo ink. It is worthy to note that the disclosed technology does not depend on the photomodulation of living tissue but creates an environment where there is little to no photomodulation of living tissue and a high amount of photomodulation in relation to the bonds of the tattoo ink.

The light beam used in the disclosed technology is generated by an ultra bright LED(s). The energy output from the ultra bright LED(s) is concentrated on a tattooed area of the recipient. The energy output generated during a removal session penetrates the epidermis of the recipient and goes through the epidermis into the dermis in which the tattoo ink is situated. The energy output is such that the light degrades the tattoo ink but does not cause any damage the surrounding tissue cells.

In one implementation, as shown in FIG. 6, a light panel 1 can house a single ultra-bright light emitting diode (LED) 2. The light panel can also have a adjustable lens 4 over the LED 2. The LED 2 can have a primary wavelength between 400-940 nm and produce a continuous and nonpulsed light. The panel 1 can have an LED intensity of about 15,000-20,000 mcd with a viewing angle of 8-30 degrees equating to ˜0.2 to 5 lumen. The light panel 1 can include controls 3 that actuate, deactuate, and regulate the light beam of the ultra bright LED. The light beam can be directly applied over the entire tattooed area, generating a pre-determined illumination (unit Lux or lx), for a specified period of time (approximately 5-30 minutes) resulting in degradation of the tattoo ink by penetrating an epidermis of the subject without overheating and/or damaging the epidermis and enters a dermis of the subject in which tattoo ink resides.

In another implementation, as shown in FIG. 1, the light panel can include a tight array of ultra-bright LEDs having an LED intensity of about 5,000-10,000 mcd with a viewing angle of 30-120 degrees equating to ˜1 to 35 lumen without the use of pulsed radiation. As more LED's are used the intensity can be lowered as well as increasing the size of the beam angle in order to distribute a certain amount of light evenly on the entire tattoo area. The array of LEDs can deliver high intensity light to skin containing tattoo ink. The array also may contain LEDs of several peak intensities to cover a wider visible spectrum output dependent on the colors of the tattoo.

The energy output from the tight array of ultra-bright LEDs can be continuously applied directly over the entire tattooed area, generating a pre-determined illumination (unit Lux or lx), for a specified period of time resulting in degradation of the tattoo ink. The time the tattooed skin is exposed to the light of the ultra bright LED and the illumination factor is dependent upon factors including the colors in the tattoo as well as the tattoo size.

The light panel 20 can include a proximal end 22 that has an ultra bright LED panel 24. The ultra bright LED panel 24 can house one or more ultra bright LEDs 26. In some embodiments, the device has a distal end 28 that has a control device 30 that has switches to actuate, deactuate, and regulate the ultra bright LED panel 24. The distal end may be configured so that the LED panel 24 and the plurality of ultra bright LED cluster probes direct the panel.

Referring to FIG. 2, a hand-held light panel 20 can be circular in shape. It is, however, understood, that the hand-held light panel 20 may be any different type or shape. Referring to FIG. 3, the LED panel 24 is slightly concave and is designed is for treating facial tattoos. The LED panel 24 may be advantageously shaped for treating facial tattoos of a person who is sitting in a chair.

Referring to FIG. 4, a block diagram shows various components that are used with an optical device constructed in accordance with the present invention are shown. In some implementations, the components are an AC power supply 32 that supplies power to an AC to DC converter 34 that is connected to a timer 36, a PCB (Printed Circuit Board) circuit 38 and ultra bright LED clusters 40 in series. The AC power supply 32 is converted to DC power supply by the AC to DC converter 34. The timer 36 that is connected in series to the converter 34 controls the time for which the ultra bright LED clusters 40 is in operation. The PCB circuit 38 is able to provide a variety of time and intensity settings for the timer 38 and ultra bright LED clusters 40. The time for which the ultra bright LED clusters are kept on may vary from case to case. Similarly, the intensity of the light produced by ultra bright LED clusters may vary and the number of ultra bright LED clusters that are in operation can be changed depending upon the requirement, e.g., size and color of the tattoo. The number of ultra bright LED clusters that are on is adjusted using the settings provided by the PCB circuit 38. The LED ultra bright clusters are configured to penetrate the outer skin layer without damaging said outer skin for effective tattoo removal. The average energy output, in a 15 minute session, can be approximately 300-600 Joules. For example, if an LED puts out ˜.004 W/cm2 at 10 cm distance and 1 W=60 J/min in 15 min 3.6 J/cm2 are produce which equates to ˜360 J for a 10×10 cm area. However, it is understood to one skilled in the art that the average energy output can also vary depending on the length of the session and output of the LED(s).

Referring to FIG. 5, FIG. 5 depicts a diagram that illustrates a use of a flexible neck in accordance with the optical device 20 in accordance with the present invention. A flexible neck 42 connects the lamp 44 containing the ultra bright LED clusters to a power board 46. The flexible neck advantageously allows the device 20 to be maneuvered and focuses the light 48 radiated by the ultra bright LED clusters on a tattooed area 50 with greater accuracy and flexibility.

In use, L-Arginine can be applied to the tattooed region before administering the LED light to assist in the fading process but is not necessary for the disclosed technology to fade a tattooed area. The L-Arginin helps create enlarged blood vessels which bring greater blood flow to the tattoo area. In addition, it creates an increase in the immune system response. These two mechanisms may help speed up the removal of the by-products of the degradation of the tattoo dyes, thus, allowing for the tattoo to fade more quickly. Additionally, an IRM (immune response modifier) compound can be applied. Specifically, IRM compounds containing L-Arginine can also increase the concentration of macrophages in the blood. Macrophages are specifically located in the lymph nodes and are white blood cells that phagocytizes necrotic cell debris and foreign material, including viruses, bacteria, and tattoo ink. The IRM compound may be selected from a group consisting of imidazoquinoline amine; a tetrahydroimidazoquinoline amine; an imidazopyridine amine; a 1,2-bridged imidazoquinoline amine; a 6,7-fused cycloalkylimidazopyridine amine; animidazonaphthyridine amine; a tetrahydronaphthyridine amine; an oxazoloquinoline amine; a thiazoloquinoline amine; an oxazolopyridine amine; a thiazolopyridine amine; an oxazolonaphthyridine amine; a thiazolonaphthyridine amine; or a 1H-imidazodimer fused to a pyridine amine, a quinoline amine, a tetrahydroquinoline amine, a naphthyridine amine, and a tetrahydronaphthyridine amine.

### Example 1

The operator places a light apparatus approximately 1 to 2 inches above a small tattooed area. (Please note that when the LED is too close to the subject's skin, e.g., less than 1 inch, the skin can (1) burn after 2-3 sittings (1 sitting=20 min under LED exposure) and/or (2) become rough due to dehydration and change in color intensity of tattooed portion is hardly visible to naked eye whereas when the LED is kept too far from the subject's skin, e.g. more than 2 inches, there is no change in color intensity of tattoo ink.) The light apparatus contains a single Edistar version 9 Warm White LED Product # ENSX-05-0707-EE-1 having 2800 Lumen @ 2000 mA/300 mA and 25° C., 222.7526 cadella@ 2000 mA/300 mA and 25° C. with a standard emission angle of 120 degrees. The tattoo area is then exposed to the continuous light generated by the ultra bright LED for 15 minutes. Depending on the distance, the illumination of the tattooed area is ˜7000 to 30000 lux. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. That is, the energy output of the ultra bright LED will break apart the bonds of the tattoo ink and cause it to be dispersed and absorbed into the body. By using this energy output, the tattoo can be removed.

### Example 2

Apply L-Arginine to a lame tattooed region and then place an LED apparatus approximately 1 to 2 inches above the tattooed area. The apparatus can contain 120 Edistar version 9 Cool White LED Product #ENSW-10-1010-EB-1 having 7000 Lumen @ 2000 mA/300 mA at 25° C. 556.8815 cadella@ 2000 mA/300 mA at 25° C. with a standard emission angle of 120 degrees clustered in twelve rows of ten LEDs each. Depending on the distance, the illumination of the tattooed area is ˜8000 to 20000 lux per LED. The tattoo area is then exposed to the continuous light generated by the clustered ultra bright LEDs for 15 minutes. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. This treatment can be applied approximately six times over a three to four month period with about two to three weeks between treatments.

### Example 3

The operator places a light apparatus approximately 1 to 2 inches above a medium-sized tattooed area. The light apparatus contains 80 Ultra-Bright White 5 mm LED 8000 mcd with viewing angle of 90 degrees clustered in eight rows of ten LEDs each. Depending on the distance, the illumination of the tattooed area is ˜3000 to 12000 lux per LED. The tattoo area is then exposed to the continuous light generated by the clustered ultra bright LEDs for 15 minutes. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. Thus, resulting in the tattoo being removed.

### Example 4

The operator applies a thin layer of 10% to 15% of L-Arginine directly to a medium-sized tattoo area. The operator then places a light apparatus approximately 1 to 2 inches above the tattooed area after L-arginine as been administered. The light apparatus contains 100 Ultra-Bright White 5 mm LED 6000 mcd with viewing angle of 100 degrees clustered in ten rows of ten LEDs each. Depending on the distance, the illumination of the tattooed area is ˜6000 to 10,000 lux per LED. The tattoo area is then exposed to the continuous light generated by the clustered ultra bright LEDs for 15 minutes. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. Thus, resulting in the tattoo being removed.

In some implementations, the disclosed technology can be a combination device for applying a treatment of light and ultrasound on a tattooed area of a subject for tattoo removal. The device can include an ultrasound device and a light panel. A control panel controls the plurality of ultra-bright LEDs and ultrasound.

During tattoo applications, dermal cells consume and store tattoo particles in vacuoles in the same manner fat cells store lipids. More specifically, tattoo ink contains carbon particles that are suspended in water. When the tattoo ink is introduced to the skin through a needle, the water diffuses. The ink itself then spreads into the surrounding tissue cells and embeds into the skin. The tattooed cells then adopt an “effective density” analogous to the way fat cells develop a lower density.

In removing the tattoos, it was found that this change in cell density can be used to as advantage. In a process called cavitation, sound waves are used to reduce the pressure of a liquid to the point where tiny bubbles of gas form. When the pressure is raised, the bubble collapses violently, generating huge pressures, albeit on a tiny scale.

Primarily, three key parameters of ultrasound-frequency, intensity, and exposure time—play influential roles in the performance and efficacy of ultrasound—mediated therapies. When used as a tattoo removal technique it was found that high frequency ultrasound at a certain intensity and pulse lengths can be used to target tattooed cells. In a preferred embodiment, an ultrasound device may use a high frequency ultrasound having a minimum frequency of 3 MHz and a maximum frequency of 10 MHz with an intensity of a minimum frequency of 12.0 W/cm2 and maximum of 25.6 W/cm2 because the effects of skin permeability begins to decrease after reaching an intensity of 21.9 W/cm2 or more.

As a result of these multiple factors, the duration of the ultrasound treatment will be modified in order to minimize any potential thermal buildup. Continuous application of ultrasound will not be used. Instead, ultrasound pulses will be implemented in order to allow tissue recovery between each pulse. Furthermore, if necessary, longer intersonication delays can be integrated into the treatment process if thermal buildup develops. Surface cooling can also be used during treatments to minimize thermal injury to the skin. Also recommended is the use of a “spiraling” motion during the treatment process. This is done in order to create a more uniform temperature throughout the treated area. It also decreases the chances of excessive thermal buildup in one specific section.

When using ultrasound, the tattooed cells may be selectively disrupted based on differences in mechanical and acoustic properties between healthy and tattooed cells. That is, different ultrasound frequencies and intensities may be used during the cavitation process to collapse tattoo cells and destroy pigment particles without damaging healthy tattoo-free tissue. The result is a technique that safely, economically, and efficiently removes at least significant portions of the ink. However, ultrasound alone will not remove all of the tattoo ink from the tattooed area.

It was found that if LED light waves where used within a specified time after the application the ultrasound, the ink could be more readily degraded and the body will more quickly rid itself of the tattoo ink. In use, it was also found that using certain wavelengths of light can destroy the bonds that hold tattoo ink together. in operation, the light device works by using the energy contained in the light beam so that the energy is absorbed by the tattoo ink dyes. This absorbed energy results in an increased stretching, vibration and bending of the bonds which hold the dye (ink) molecules together. Ultimately, these bond stresses cause bond deformation with resulting bond failure.

In use, the ultrasound device produces high-frequency ultrasound waves. The high frequency ultrasound waves have a frequency of about 5 MHz and an intensity of about 19.8 W/cm2. The ultrasound sound waves are administered in pulses in order to allow tissue recovery between each pulse. These waves are applied directly to the tattooed area for a specified period of time (approximately 10 minutes) resulting in cavitation of tattooed cells.

The light panel can house houses one or more ultra-bright light emitting diodes (LEDs). The LED(s) have a wavelength between 660-700 nm resulting in (a) minimal absorption by melanin and hemoglobin of the subject and (b) little to no heat being generated on the epidermis of the subject while generating heat on the tattoo ink thereby causing increased molecular motion and bond deformation of the tattoo ink and produces a continuous light. The ultra-bright LED(s) is approximately equal to size of the tattooed area and has an energy output of about 88 joules per square inch without the use of pulsed radiation. The light can be directly applied over the entire tattooed area for a specified period of time (approximately 5-15 minutes) resulting in degradation of the tattoo ink and penetrates an epidermis of the subject without damaging the epidermis by overheating and enters a dermis of the subject in which tattoo ink resides.

### Example 5

High frequency ultrasound having a frequency of 5 MHz and an intensity of 19.8 W/cm2 is applied to a tattooed area treated with an ultrasound gel for 10 minutes. The ultrasound will cause cavitation of the tattooed cells. After the ultrasound has been applied, the operator will wipe off the ultrasound gel, wait approximately two minutes for the patient's skin to cool, apply L-Arginine to the tattooed region and then place the LED apparatus approximately 1 to 2 inches above the tattooed area. The apparatus contains one or more ultra bright LEDs. The tattoo area is then exposed to the continuous light generated by the LED(s) for 15 minutes. The average energy output, in this 15 minute session can be 480 Joules. During this period of time, the light penetrates through the epidermis and into the dermal layer in which the tattoo resides. The absorption of the energy by the tattoo ink results in both heat generated in the ink molecules by molecular vibration and molecular bond deformation by vibration, stretching and bending. This dual treatment can be applied approximately six times over a three to four month period with about two to three weeks between treatments.

The advantages of this combination therapy is that the cavitation causes the tattooed cells to dispel the ink and then once the ink is exposed without the protection of the cell membrane the LED light will further break the bonds of the ink so the body may more readily dispose of the ink naturally.

The foregoing Detailed Description is to be understood as being in every respect illustrative, but not restrictive, and the scope of the disclosed technology disclosed herein is not to be determined from the Detailed Description, but rather from the claims as interpreted according to the full breadth permitted by the patent laws. It is to be understood that the implementations shown and described herein are only illustrative of the principles of the disclosed technology and that various modifications can be implemented without departing from the scope and spirit of the disclosed technology.

1. An apparatus comprising:
a light panel housing at least one ultra-bright light emitting diode (LED), the LED having an intensity of about 15,000-20000 mcd and a viewing angle of 8-30 degrees and a primary wavelength between 400-940 nm, the intensity, the viewing angle and the primary wavelength being dependent on a size and a color of a tattooed area, the light panel continuously applying an energy output from the light panel directly over the tattooed area for a specified period of time resulting in degradation of the tattoo ink, wherein the light panel is used to apply a treatment of light on a tattooed area of a subject for tattoo removal, the light panel generating a light beam that penetrates an epidermis of the subject without damaging the epidermis by overheating and enters a dermis of the subject in which tattoo ink resides and results in (a) minimal to no absorption by melanin and hemoglobin of the subject and (b) little to no heat being generated on the epidermis of the subject while generating heat on the tattoo ink thereby causing increased molecular motion and bond deformation of the tattoo ink.

2. The apparatus of claim 1 wherein the light beam is approximately equal to or greater than a size of the tattooed area.

3. The apparatus of claim 1 wherein the specified period of time is approximately 5-30 minutes.

4. The apparatus of claim 1 further comprising;
a control panel for controlling the light panel, wherein the control panel includes a timer for controlling the duration of a treatment.

5. The apparatus of claim 4 wherein the control panel includes a printed circuit board for controlling the timer and an intensity of the light panel.

6. The apparatus of claim 1 wherein L-Arginine is applied to the tattooed area before treatment begins.

7. The apparatus of claim 1 wherein an immune response modifier compound is applied to the tattooed area before treatment begins.

8. The apparatus of claim 1 wherein an immune response modifier compound containing L-Arginine is applied to the tattooed area before treatment begins.

9. A method comprising the steps of:
positioning a light panel a specific distance from a tattooed area, the light panel housing at least one ultra-bright light emitting diode (LED), the LED having an intensity of about 15,000-20000 mcd and a viewing angle of 8-30 degrees and a primary wavelength between 400-940 nm, the intensity, the viewing angle and the primary wavelength being dependent on a size and a color of a tattooed area,;
generating a light beam from the light panel, the light beam penetrating an epidermis of a subject without damaging the epidermis by overheating and enters a dermis of the subject in which tattoo ink resides and results in (a) minimal absorption by melanin and hemoglobin of the subject and (b) little to no heat being generated on the epidermis of the subject while generating heat on the tattoo ink;
continuously applying the energy output from the light panel directly over the tattooed area for a specified period of time; and
causing increased molecular motion and bond deformation of the tattoo ink resulting in degradation of the tattoo ink and effectively removing the tattoo.

10. The method of claim 9 further comprising the step of:
applying L-arginine to said tattooed skin region.

11. The method of claim 10 wherein the step of applying further includes the step of:
mixing L-arginine with an immune response modifier to obtain a specific concentration thereof.

12. The method of claim 11 wherein said immune response modifier is a chemical selected from the group consisting of:
imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydronaphthyridine amine, an oxazoloquinoline amine, a thiazoloquinoline amine, an oxazolopyridine amine, a thiazolopyridine amine, an oxazolonaphthyridine amine, a thiazolonaphthyridine amine, and a 1H-imidazodimer fused to a pyridine amine, a quinoline amine, a tetrahydroquinoline amine, a naphthyridine amine, and a tetrahydronaphthyridine amine.

13. The method of claim 9 wherein the specified period of time is five to thirty minutes.

14. An apparatus for applying a treatment of light on a tattooed area of a subject for tattoo removal, the apparatus comprising:
an LED panel, the LED panel including at least one ultra bright LED, the LED having an intensity of about 5,000-20000 mcd and a viewing angle of 8-130 degrees and a primary wavelength between 400-940 nm, the intensity, the viewing angle and the primary wavelength being dependent on a size and a color of the tattooed area, the LED panel producing a continuous light beam, wherein the apparatus generates a light beam that penetrates an epidermis of the subject without damaging the epidermis by overheating and enters a dermis of the subject in which tattoo ink resides.

15. The apparatus of claim 14 wherein the light beam is approximately equal to size of the tattooed area.

16. The apparatus of claim 14 wherein the specified period of time is approximately 5-15 minutes.

17. The apparatus of claim 14 wherein the ultra-bright LED panel is slightly concave.

18. The apparatus of claim 14 further comprising:
a control panel for controlling the at least one ultra-bright LED, wherein the control panel includes a timer for controlling the duration of a treatment.

19. The apparatus of claim 18 wherein the control panel includes a printed circuit board for controlling the timer and an intensity of the LED panel.

20. The apparatus of claim 10 wherein L-Arginine is applied to the tattooed area before treatment begins.

21. An apparatus for applying a treatment of light and ultrasound on a tattooed area of a subject for tattoo removal, the apparatus comprising:
an ultrasound device, the ultrasound device producing a high-frequency ultrasound waves, the ultrasound device applying the high-frequency ultrasound waves directly to the tattooed area for a first specified period of time resulting in cavitation of tattooed cells; and
a light panel housing at least one ultra-bright light emitting diode (LED), the panel producing a continuous light, the at least one ultra-bright LED continuously applying the energy output from the at least one ultra-bright LED directly over the entire tattooed area for a second specified period of time resulting in degradation of the tattoo ink.

22. The apparatus of claim 21 further comprising:
a control panel for controlling the at least one ultra-bright LED and ultrasound.

23. The apparatus of claim 21 wherein the light penetrates an epidermis of the subject without damaging the epidermis by overheating and enters a dermis of the subject in which tattoo ink resides.

24. The apparatus of claim 21 wherein the at least one LED has a wavelength between 660-700 nm resulting in (a) minimal absorption by melanin and hemoglobin of the subject and (b) little to no heat being generated on the epidermis of the subject while generating heat on the tattoo ink thereby causing increased molecular motion and bond deformation of the tattoo ink.

25. The apparatus of claim 21 wherein the light generated by the at least one ultra-bright LED is approximately equal to size of the tattooed area.

26. The apparatus of claim 21 wherein the at least one ultra-bright LED has an energy output of about 88 joules per square inch without the use of pulsed radiation.

27. The apparatus of claim 21 wherein the second specified period of time is approximately 5-15 minutes.

28. The apparatus of claim 21 wherein the high frequency ultrasound device administers the ultrasound sound waves with a frequency of about 5 MHz and an intensity of about 19.8 W/cm2.

29. The apparatus of claim 21 wherein the first specified period of time is approximately 10 minutes.

30. The apparatus of claim 1 wherein the ultrasound sound waves are administered in pulses in order to allow tissue recovery between each pulse.

31. A method for removing tattoos comprising the steps of:
applying an ultrasonic gel to a tattooed skin region;
positioning an ultrasonic device in direct contact with the tattooed area;
exposing the tattooed skin region to high-frequency ultrasound waves for a first specified period of time resulting in cavitation of tattooed cells;
cooling the tattooed area;
applying L-argirine to a tattooed skin region,
positioning an optical device including at least one LED at a specific distance from said tattooed skin region, and
exposing said tattooed skin region to continuous LED energy without pulsing in the range of 660 nm to 700 nm wavelengths for a timed interval.

32. The method of claim 31 wherein the light penetrates an epidermis of the subject without damaging the epidermis by overheating and enters a dermis of the subject in which tattoo ink resides.

33. The method of claim 31 wherein the at least one LED has a wavelength between 660-700 nm resulting in (a) minimal absorption by melanin and hemoglobin of the subject and (b) little to no heat being generated on the epidermis of the subject while generating heat on the tattoo ink thereby causing increased molecular motion and bond deformation of the tattoo ink.

34. The method of claim 31 wherein the light generated by at least one ultra-bright LED is approximately equal to size of the tattooed area.

35. The method of claim 31 wherein the at least one ultra-bright LED has an energy output of about 88 joules per square inch without the use of pulsed radiation.

36. The method of claim 31 wherein the second specified period of time is approximately 5-15 minutes.

37. The method of claim 11 wherein the high frequency ultrasound device administers the ultrasound sound waves with a frequency of about 5 MHz and an intensity of about 19.8 W/cm2.

38. The method of claim 11 wherein the first specified period of time is approximately 10 minutes.

39. The method of claim 11 wherein the ultrasound sound waves are administered in pulses in order to allow tissue recovery between each pulse.
